(19) United States
(12) Patent Application Publication
(10) Pub. No.: US 2017/0120071 A1
(43) Pub. Date: May 4, 2017

(54) SYSTEM, APPARATUS, AND METHOD OF PROVIDING TATTOO FADING AND REMOVAL

(71) Applicant: DABNEY PATENTS, L.L.C., Georgetown, TX (US)

(72) Inventor: Paul Dabney, Georgetown, TX (US)

(73) Assignee: DABNEY PATENTS, L.L.C., Georgetown, TX (US)

(21) Appl. No.: 15/337,245

(22) Filed: Oct. 28, 2016

Related U.S. Application Data
(60) Provisional application No. 62/248,721, filed on Oct. 30, 2015.

(51) Int. Cl. A61N 5/06; A61K 8/42; A61K 8/22; A61K 8/36; A61Q 1/14; A61K 8/60

(52) U.S. Cl. CPC A61N 5/0616; A61Q 1/145; A61K 8/602; A61K 8/22; A61K 8/361; A61K 8/42; A61N 5/062; A61K 2800/91; A61K 2800/81; A61N 2005/0626; A61N 2005/063

(57) ABSTRACT

A retainer, system and method of tattoo removal including treating a tattooed region of skin with an oxidizing or bleaching solution activated by certain wavelengths of light may be provided. The method of tattoo removal may include applying a layer of removal solution to a tattooed region of skin; regulating a temperature of the layer of removal solution with a temperature control element; and exposing the layer of removal solution to a light source of pre-determined wavelength. Light of certain wavelengths may improve or “supercharge” the effects of certain pharmaceuticals or target chemicals and agents, creating a synergistic effect that may break down tattoo ink molecules and eliminate the appearance of tattoos in the skin of humans or other animals.

### CROSS REFERENCE TO RELATED APPLICATIONS

This application claims the benefit of the filing date of U.S. Patent Application No. 62/248,721 filed Oct. 30, 2015, which is incorporated herein by reference in its entirety.

### BACKGROUND

Tattooing is a form of body modification involving the injection of ink particles into the papillary and superficial dermis, thereby causing a visible change in skin pigmentation. Upon initial injection of a needle into the skin, ink particles are dispersed throughout a homogenized upper layer of skin (epidermis) into the underlying dermis. As the skin heals, the damaged epidermis sheds and eliminates surface pigment. Deeper within the skin, granulation tissue forms and allows the growth of collagen; this mends the upper dermis, where pigment remains trapped within fibroblasts in a layer just below the epidermis/dermis boundary.

The practice of permanent tattooing has become so widespread that an estimated 45 million Americans have at least one tattoo. However, with the tremendous growth of the industry comes an increasing number of people seeking tattoo removal on a more frequent basis. Although tattoos are no longer considered permanent and irreversible marks on the skin, there is not one universally practiced tattoo removal treatment. Common removal techniques once included physical destruction of the skin layers, such as dermabrasion, chemical destruction, surgical excision, and cryotherapy. More modern practice employs pulsed laser irradiation. Although some of these treatments may be effective, they are often painful and cost-prohibitive. These procedures also require prolonged recovery time, involve a high risk of scarring and skin discoloration and ultimately produce unpredictable results.

### SUMMARY

Exemplary embodiments described herein generally relate to a cosmetic and therapeutic treatment including the enhancement of chemical reactions with light and more specifically to cosmetic and medical treatments of light in combination with oxidizing or bleaching solution for tattoo removal. Light of certain wavelengths has been demonstrated to improve or “supercharge” the effects of certain pharmaceuticals or target chemicals and agents, creating a synergistic effect that may break down tattoo ink molecules and may fade or eliminate tattoos in the skin of humans or other animals.

According to an exemplary embodiment, a retainer for cosmetic and therapeutic treatment may retain an oxidizing or bleaching solution against a tattooed region of tissue. The retainer may include a number of heating elements, and a number of oxidizing or bleaching solution delivery elements that may deliver an oxidizing or bleaching solution to the area to be treated. A number of light terminations rigidly connected to the retainer for cosmetic and therapeutic treatment may be connected to a light source through a fiber optic cable.

According to another exemplary embodiment, a method of tattoo removal that includes treating a tattooed region of skin with an oxidizing or bleaching solution activated by certain wavelengths of light may be provided. The method of tattoo removal may include applying a layer of removal solution to a tattooed region of skin; regulating a temperature of the layer of removal solution with a temperature control element; and exposing the layer of removal solution to a light source of pre-determined wavelength.

### BRIEF DESCRIPTION OF THE DRAWINGS

Advantages of exemplary embodiments of the system, retainer and method of providing cosmetic and therapeutic treatment will be apparent from the following detailed description of the exemplary embodiments. The following detailed description should be considered in conjunction with the accompanying figures in which:

FIG. 1 depicts an exemplary embodiment of a solution retainer according to the present invention;

FIG. 2 depicts a diagram showing another exemplary embodiment of a solution retainer according to the present invention;

FIG. 3 depicts an exemplary embodiment of a full body suit retainer;

FIG. 4 depicts an exemplary embodiment of a bowl retainer; and

FIG. 5 depicts an exemplary embodiment of a hand held light emitting device.

### DETAILED DESCRIPTION

Aspects of the present invention are disclosed in the following description and related figures directed to specific exemplary embodiments of the invention. Those skilled in the art will recognize that alternate exemplary embodiments may be devised without departing from the spirit or the scope of the claims. Additionally, well-known elements of exemplary embodiments of the invention will not be described in detail or will be omitted so as not to obscure the relevant details of the invention.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”. “embodiments” or “invention” do not require that all embodiments of the invention include the discussed feature, advantage, or mode of operation.

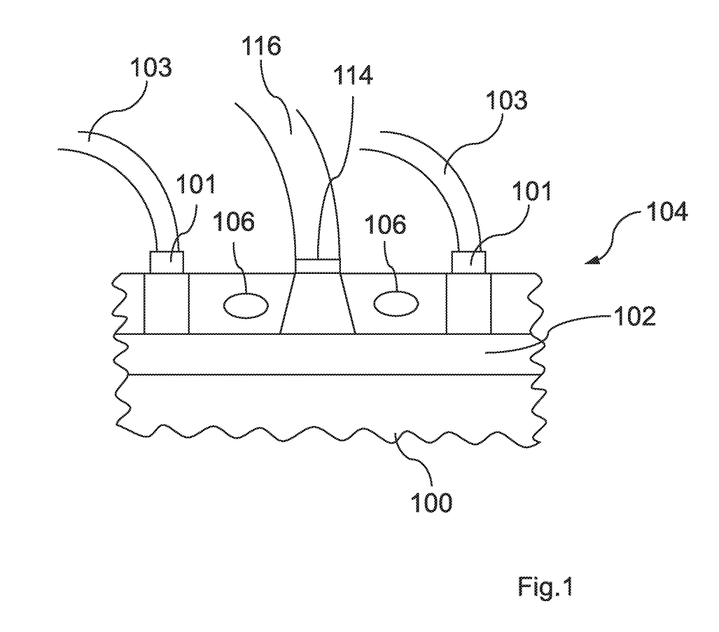

In an exemplary embodiment, and generally referring to FIGS. 1-5, a system, retainer, and method of providing cosmetic and therapeutic treatment for tattoo removal may be provided. During treatment, a retainer to hold oxidizing or bleaching solutions in contact with tattooed regions of tissues may be used while the tissues and solutions may be simultaneously exposed to certain wavelengths of light. The oxidizing or bleaching solutions may be activated or combined with light of a predetermined wavelength and may eliminate or reduce the visibility of tattoos with a greater efficiency than the solution acting alone. An exemplary embodiment of this system may create a synergistic effect between certain wavelengths of light and oxidizing or bleaching solutions that, when applied to tissues, may degrade or eliminate tattoo pigments residing in a dermal layer of skin.

In another exemplary embodiment, the retainer may include a retainer for cosmetic and therapeutic treatment that emits certain wavelengths of light into the solution. When a specific composition of solution is combined with certain wavelengths of light and applied to tissues, a synergistic effect may be created that may chemically react with the tattoo ink pigments, thereby eliminating or fading the tattoo. In an exemplary embodiment, the ink molecules or molecular structure of tattoo pigments may be altered by reaction with the specific light and solution. Once the ink molecules or molecular structure of the tattoo pigments are altered, they may lose their physical properties, such as size and color, and may be broken down in substructures small enough for the body to eliminate. It may be appreciated that tattoo ink pigments may include commercially available tattoo ink, homemade inks and any pigment not primarily intended for tattooing, as may be understood by a person having ordinary skill in the art.

In another exemplary embodiment, the retainer for cosmetic and therapeutic treatment 104 may hold solutions in contact with tissues 100, such as skin or flesh, while the tissues 100 and solutions 102 may be simultaneously exposed to certain wavelengths of light. The retainer for cosmetic and therapeutic treatment 104 may include at least one covering tray that may emit light for treatments creating a synergistic oxidizing or bleaching effect between an oxidizing or bleaching solution and a light of certain predetermined wavelengths. The resulting synergistic effect may be greater than the effect of an oxidizing or bleaching solution or the light acting alone. The retainer 104 may be applied to one or more human or animal tissue areas. A light source may shine from a light emitting device or from the end of a fiber optic cable. The fiber optic cable may have one side exposed down its length. An oxidizing or bleaching solution 102 may fill a tissue-covering tray. In an exemplary embodiment, the light source may be blue, or another predetermined wavelength of light that may supercharge the solution, with an exposure from, but not limited to, a few second to several minutes, as desired. It may be appreciated that any desired wavelength or exposure time may be applied. It may also be appreciated that the treatment may be performed in multiple sessions, over a period of hours, days or weeks at a time until the desired reduction in tattoo pigment is acquired.

In an exemplary embodiment, an oxidizing or bleaching solution 102 may be used without a reservoir. The retainer for cosmetic and therapeutic treatment 104 may include devices to inject the solutions 102 at pre-determined depth into the skin or tissue 100. Alternatively, the oxidizing or bleaching solution 102 may be applied directly to the body and exposed to light by a retainer in the form of a fiber optic wand with a number of light emitting fibers. Further exemplary embodiments of a carrier or reservoir may be the solution 102 itself or a gel. This gel may be applied to a body with an applicator and exposed to the synergizing light by the same applicator or a different device. The synergizing light may create an oxidizing or bleaching effect between an oxidizing or bleaching solution and a light of certain predetermined wavelengths. The resulting oxidizing or bleaching effect may be greater than the effect of an oxidizing or bleaching solution or the light acting alone. It may be appreciated that the oxidizing or bleaching solution 102 may be delivered by any desired expedient, including, but not limited to, reservoirs, bandages, gels, solutions, head coverings, wraps, socks, stockings, hats, helmets, mitts, and suits. In another exemplary embodiment, the retainer for cosmetic and therapeutic treatment may be a light emitting sponge that can apply and hold the solution on the surface of the tissue.

In an exemplary embodiment illustrated in FIG. 1, a layer of oxidizing or bleaching solution 102 may be held between the tissue 100 and the retainer for cosmetic and therapeutic treatment 104. A tattoo (not shown) may be located in the dermal layer of skin, below the tissue 100 surface. In an exemplary embodiment, one end of a number of fiber optic cables 103 may be rigidly attached to the retainer for cosmetic and therapeutic treatment 104 through a number of fiber optic connection interfaces or plugs 101. A second end of each fiber optic cable 103 may be rigidly connected to any desired source including, but not limited to, a light emitting diode (LED) and laser. The activation of the oxidizing or bleaching solution by a light of a pre-determined wavelength may create a synergistic oxidizing or bleaching effect. The resulting synergistic effect may be greater than the effect of oxidizing or bleaching solution or the light acting alone. In an exemplary embodiment, the retainer 104 may include temperature control elements 106 that warm and further activate the oxidizing or bleaching solution layer 102. It may be further appreciated that the temperature control elements 106 may also cool the oxidizing or bleaching solution, or otherwise regulate the temperature of the oxidizing or bleaching solution, as desired. The temperature control elements may include a fan, thermoelectric device, electric coil, or any other suitable temperature control element as may be understood by a person having ordinary skill in the art. The temperature control elements may control the temperature within a predetermined range of degrees; for example, an upper temperature limit may never exceed a point in which the heat could cause tissue damage, pain or scalding. The lower temperature limit may be variable. In the event that the oxidizing or bleaching solution may be refrigerated or stored at a temperature lower than desired, additional temperature elements may heat the solution prior to application on the skin. This may be accomplished with an oven, warm bath, microwave, or any conventional heating source as may be appreciated by a person having ordinary skill in the art. In a further exemplary embodiment, a number of oxidizing or bleaching solution delivery elements 114 may be rigidly connected to the retainer 104 for cosmetic and therapeutic treatment 104 and to a number of tubes 116.

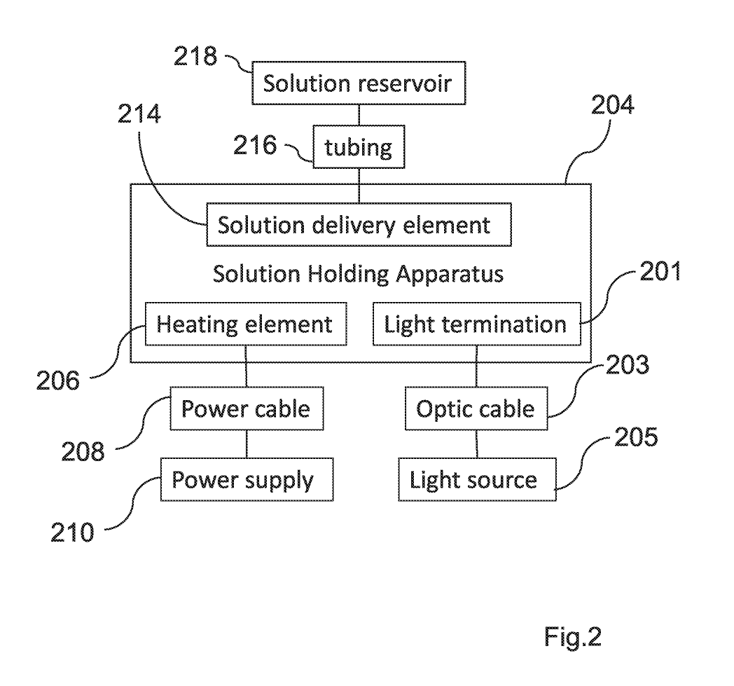

Now referring to FIG. 2, an exemplary embodiment of a retainer for providing cosmetic and therapeutic treatment may be provided. The retainer for cosmetic and therapeutic treatment 204 may house a number of heating elements 206 connected to a power supply 210 by a power cable 208. In another exemplary embodiment, a retainer for cosmetic and therapeutic treatment 204 may house a number of light terminations 201 that may be connected to a light source 205 by optic cable 203. In a further exemplary embodiment, a retainer for cosmetic and therapeutic treatment 204 may house an oxidizing or bleaching solution delivery element 214 connected to an oxidizing or bleaching solution reservoir 218 though a tubing 216.

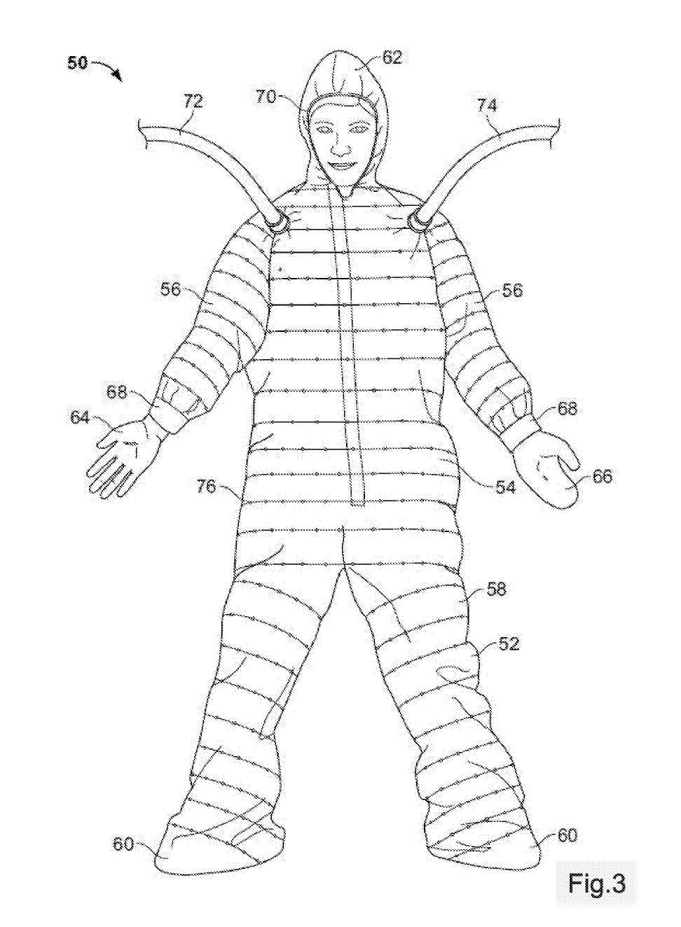

According to another exemplary embodiment, and generally referring to FIG. 3, a full body suit retainer may be provided. A full body suit with tubing may carry a light enhanced oxidizing or bleaching mist or solution to a user wearing the suit. An embodiment of a suit may include a one-piece garment that generally retains air around the user's body. A full body suit may be used while treating a tattoo extending over a significant part of the body. Exemplary embodiments of a full body suit may include a portion that covers at least a user's torso, as desired. Exemplary embodiments may further include portions to cover a user's legs, feet, arms, head, or hands, as desired. Further exemplary embodiments may have collars, cuffs, or elastic portions that may fit against the user to help retain the oxidizing or bleaching solution, as desired. The portions may cooperate to provide a garment that may retain the oxidizing or bleaching solution within the suit when the suit is worn. A fiber optic cable may wrap around the inside of the suit. A full body suit may include sleeves with gloves or mittens, pants with feet, and a hood with fiber optic cable spaced along the inner fabric of the suit, or otherwise within or on the fabric.

In an exemplary embodiment, gloves, mittens, or feet may help retain the oxidizing or bleaching solution within the suit, and may be integrated with the rest of the suit or may be removable. Exemplary embodiments of integrated portions may include fiber optic cable, similar to the rest of the suit. Exemplary embodiments of removable portions may lack fiber optic cable, or may include fiber optic cable that can be connected to the fiber optic cable in the sleeves or pants, as desired. The fiber optic cable may have light terminations spaced along the fiber optic cable inside the suit. The fiber optics may irradiate an oxidizing or bleaching solution with a light of certain predetermined wavelengths and may create a synergistic oxidizing or bleaching effect. The resulting synergistic effect may be greater than the effect of an oxidizing or bleaching solution or the light acting alone. An exemplary embodiment may include a heating element inside the bodysuit; such heating wires may be embedded in the fabric of the suit.

In a further exemplary embodiment, the body area covering may include an input tube or input tubing to carry an oxidizing or bleaching solution into the covering, an area within the covering to circulate the oxidizing or bleaching solution against the user, and an exhaust tube or exhaust tubing to allow air and the oxidizing or bleaching solution out of the covering. Exemplary embodiments of tubing may include an input tube on one side of the covering and an output tube on the opposite side, such as input and output tubes on opposite shoulders.

In an exemplary embodiment, a user may put on the garment, add oxidizing or bleaching solutions using the input tube, allow the mist to circulate within the suit, and turn on the light source. The user may simultaneously input and output oxidizing or bleaching solutions to circulate through the suit. The user may power on the heating element to warm the mist and/or otherwise activate or control any portion of the suit, as desired.

Further to the above, another exemplary embodiment of the cosmetic and therapeutic treatment retainer may be illustrated in FIG. 3. An embodiment of a medical device 50 may include a body area covering 52 having an integrated torso portion 54, sleeves 56, pants 58, feet 60, and a hood 62, and removable gloves 64 or mittens 66. The sleeves 56 may have cuffs 68 to tighten and/or form a seal against the user's wrists. The hood 62 may have elastic portions 70 to tighten against the user's face. Further exemplary embodiments may include an input tube 72, such as one located on shoulder of the torso portion 54, and an exhaust tube 74 on the opposite shoulder of the torso portion 54. Exemplary embodiments may further include a fiber optic cable 76 spaced along the inner fabric of the suit 52. Fiber optic cable 76 may be coupled to the suit 52 in any desired fashion.

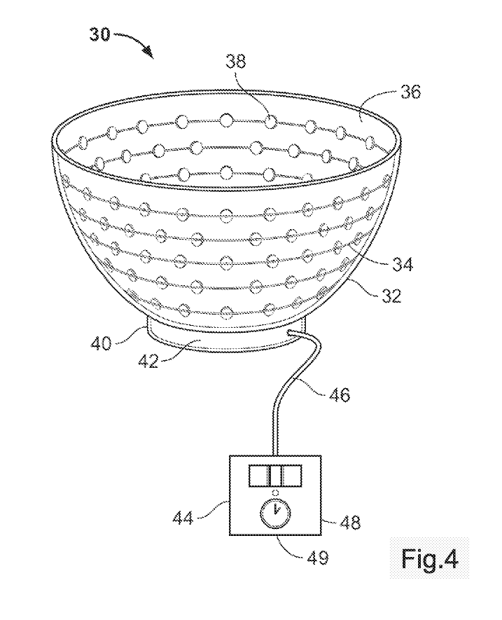

In further exemplary embodiment, and generally referring to FIG. 4, the retainer may be in the shape of a container and may apply an oxidizing or bleaching solution that may be enhanced with light of a predetermined wavelength. An exemplary embodiment may include a bowl, such as a round, open-top container that retains an oxidizing or bleaching solution. The bowl may be made of generally watertight material so it can retain liquid solution. A fiber optic cable may wrap all around the inside surface of the bowl, with light terminations spaced along the fiber optic cable. The fiber optic cable may have a number of light terminations so that an oxidizing or bleaching solution in the bowl is illuminated with light from the cable so as to substantially surround a tattooed region of tissue immersed in the bowl. An exemplary embodiment may include a heating element at the bottom of the bowl. The bowl may have a stand or base, and the heating element may be located in the base, or any other location, as desired. The fiber optic cable may connect through a connection cable to a light source. The light source may have an on/off switch and may have a timer control. Embodiments may include a control box that includes the light and heating power source, such as batteries or a connection to a power outlet. Embodiments of a control box may also include controls for turning the light or heater on or off, setting of timers, and status display. A control box may include receptacles or cables for interfacing with the rest of the retainer, such as a combined interface for both fiber optics and heater power.

In an exemplary embodiment, the oxidizing or bleaching solution may be warmed in the bowl. An object or a body part such as a hand, a foot, or a finger may be placed into the oxidizing or bleaching solution, and the light source may be turned on. The light source may interact with the oxidizing or bleaching solution and may create a synergistic oxidizing or bleaching effect. The resulting synergistic effect may be greater than the effect of an oxidizing or bleaching solution or the light acting alone. The user may utilize a control box, or any other wired or wireless controller, to turn on or off, or set timers for, the light source, the heater power, or both. It may be appreciated that any desired functions may be managed and timed by the control box.

As depicted in exemplary FIG. 4, an embodiment of a medical device 30 may include a bowl 32 with a number of fiber optic cables 34 that may wrap around an inside surface 36 of the bowl 32. The number of fiber optic cable 34 may have any number of light terminations 38. Exemplary embodiments may have a base 40, which may contain a number of heating elements 42. The fiber optic cable 34 may connect to a number of light sources 44 through a number of fiber optic connection cables 46. The number of light sources 44 may include an off/off switch 48 or a timer control 49.

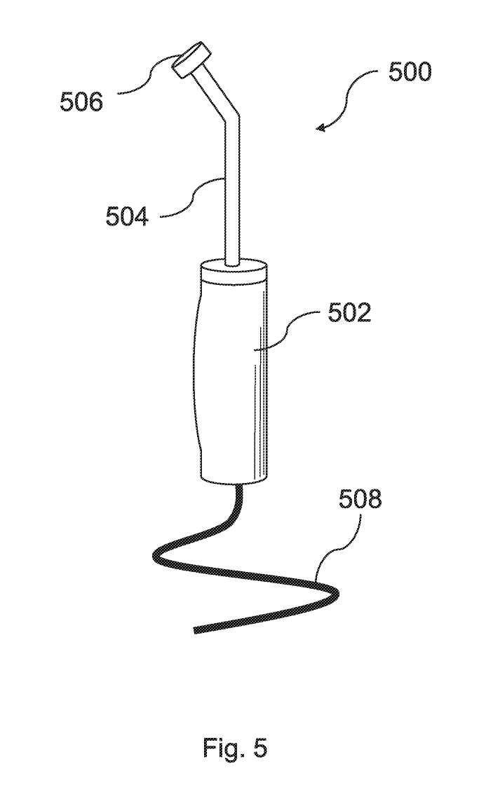

Now referring to exemplary FIG. 5, an exemplary embodiment of a hand held light emitting device 500 may be provided. The hand held light emitting device 500 may include a handle portion 502 rigidly connected to the first end of a neck 504. The second end of the neck 504 may be rigidly connected to a head portion 506 housing the light source outlet. In an exemplary embodiment, the light source may be situated in the handle portion 504 and the light may be guided through the neck by optical elements including, but not limited to, fiber optic and mirrors. It may be further appreciated that the neck 504 may be flexible and orientable. The light source may produce a light at a wavelength of about 360 nm to about 600 nm. In an exemplary embodiment, hand held light emitting device 500 may be used to irradiate an oxidizing or bleaching solution, thus creating a synergistic reaction with an oxidizing or bleaching effect greater than the light or the solution acting alone. In an exemplary embodiment, the hand held light emitting device 500 may be powered by electric power. Power sources may include, but may not be limited to, batteries, rechargeable batteries, and grid power supply. The handle portion 502 may house any necessary elements required to provide the current and intensity required by the light source, such as electrical transformers and electronic components. In a further exemplary embodiment, the hand held light emitting device 500 may be powered with batteries and may optionally require the power supply cable 508.

In an exemplary embodiment, a retainer may be a mist or gel providing the oxidizing or bleaching agent to the tattooed tissue creating a synergistic oxidizing or bleaching effect between an oxidizing or bleaching solution and a light of certain predetermined wavelengths and may be in combination with one or more of a topical antibiotic, topical anesthetic, nicotinic acid, nicotinamide, antimicrobials, salicylic acid, sulfur, retinoids such as 6[3-(1-adamantyl)-4-methoxy-phenyl] naphthalene-2-carboxylic acid, glycolic acid, tretinoin, borax, and additional chemicals useful in the method.

In other exemplary embodiments, the system, retainer, and method of providing cosmetic and therapeutic treatment may utilize blue light, or another predetermined wavelength of light that may supercharge the solution, with an exposure from about a few second to a few minutes. In a further exemplary embodiment, the oxidizing or bleaching solution may be a hydrogen peroxide (H₂O₂) solution, such as a gel, with concentration of about 0.3 mM or any concentration of solution that may be suitable as an oxidizing or bleaching agent. In an exemplary embodiment, tattoo ink that may be exposed to hydrogen peroxide alone may fade or lighten. Similarly, a 20 seconds irradiation with light of the wavelength from 360 nm to 500 nm may lighten or fade a tattoo it may be exposed to. However, hydrogen peroxide in combination with irradiation with a light of 360 nm to 500 nm may exhibit a synergistic reaction that lightens or fades a tattoo to a greater extent than if it were exposed to either hydrogen peroxide or light alone. Solution of hydrogen peroxide may work best at a temperature of about 57 degrees Celsius. It may be appreciated that other chemicals may have different preferred temperatures.

In an exemplary embodiment, and generally referring to FIGS. 1-6, topical solutions, including, but not limited to, hydrogen peroxide, carbamide peroxide, benzoyl peroxide, and other chemicals deemed effective may be delivered in various organic vehicles or carriers. Exemplary embodiments of carriers may include a combination of ethyl alcohol and propylene glycol in which the active ingredient may present in the range of from about 0.001% to about 50% by volume of the carrier. The pH of the solution may be adjusted so that tissue sensitivity may be minimized while the effectiveness of the solution may be optimized. In an exemplary embodiment, the temperature of the solution may be adjusted to increase or optimize its effectiveness. In an exemplary embodiment, additional oxidizing or bleaching agents may be used to increase the effectiveness of the treatment. The solution may be exposed to light in a wavelength of about 360 nm to about 600 nm or any other wavelength that proves effective for a certain time that may range from about 1 second to about 60 seconds.

In an additional exemplary embodiment, topical solutions of peroxide compounds may include hydrogen peroxide and/or carbamide peroxide and/or benzoyl peroxide in various organic carriers in concentrations that may range from about 0.001% to about 50% by volume of carrier.

In an exemplary embodiment, the oxidizing or bleaching solution may be incorporated into various vehicles or carriers including solutions, lotions, creams, gels, mists, pastes and ointments along with one or more of the following ingredients: nicotinic acid or nicotinamide that may be present in concentrations from about 0.001% to 30% by volume of the carrier.

In an exemplary embodiment, the oxidizing or bleaching solution may include erythromycin base in concentrations from about 0.001% to about 30% by volume of the carrier.

In another exemplary embodiment, the oxidizing or bleaching solution may contain clindamycin phosphate methyl 7-chloro-6,7,8-trideoxy-6-(1-5 methyl-trans-4-propyl-L-2pyrrolidinecarboxamido)-1-thio-L-threo-α-D-galacto-octopyranoside 2-(dihydrogen phosphate) with a concentration from about 0.001% to 30% by volume of carrier.

In another exemplary embodiment, the oxidizing or bleaching solution may contain tetracycline hydrochloride in concentrations of from about 0.001% to 30% by volume of the carrier; retinoids such as 6-[3-(1-adamantyl)-4-methoxy-phenyl] naphthalene-2-carboxylic acid; It may be appreciated that the oxidizing or bleaching solution may contain any desired compounds deemed effective in removing or fading tattoo ink.

In still other exemplary embodiments, the oxidizing or bleaching solution carriers may include combinations of ethyl alcohol and propylene glycol, surface-active agents such as lauryl ethers and lauryl esters, and other carriers effective for the desired purposes.

In other exemplary embodiments, the oxidizing or bleaching solution may include a light activated pigment that may fluoresce when exposed to the wavelength of light used in the treatment. This pigment may indicate to the user that the synergistic effect may be occurring.

This synergistic oxidizing or bleaching effect may be greater than the solution oxidizing or bleaching effect or the light's oxidizing or bleaching effect if they were used separately. The light may be applied to the irradiated area by a LED device exposing the entirety of the tattooed area or a portion of the tattooed area.

The irradiated area may be exposed to a 10 watt light source emitting at wavelengths from about 410 nm to about 500 nm thus creating a synergistic effect between the solution and the light causing a greater reduction and fading of tattoo ink than with the light or with the solution acting alone. The retainer may have a number of terminations and may be dimensioned so as to enable the targeting areas of various sizes. However, the diameter of the termination may have any desired value. The exposure time of the light may be about 60 seconds. However, it may be appreciated that any desired duration of treatment may be selected.

In another exemplary embodiment, a solution containing chemical species including, but not limited to, about 15% carbamide peroxide, about 2.5% clindamycin phosphate (Methyl 7-chloro-6,7,8-trideoxy-6-(1-methyl-trans-4-propyl-L-2-pyrrolidinecarboxamido)-1thio-L-threo-α-D-galacto-octopyranoside 2-(dihydrogen phosphate)) and tretinoin may be combined in a gel-form carrier. The tattooed area may be exposed to a light of a wavelength from about 410 nm to about 500 nm by a number of light terminations that would expose an area with a diameter of about 30 centimeters. The tattooed area and solution may be exposed to this certain wavelength of light for about 30 seconds. The synergistic effect of the light and solution that may be warmed may be greater than the effect of the light or the solution acting individually.

The foregoing description and accompanying figures illustrate the principles, preferred exemplary embodiments, and modes of operation of the invention. However, the invention should not be construed as being limited to the particular exemplary embodiments discussed above. Additional variations of the exemplary embodiments discussed above will be appreciated by those skilled in the art.

Therefore, the above-described exemplary embodiments should be regarded as illustrative rather than restrictive. Accordingly, it should be appreciated that variations to those exemplary embodiments can be made by those skilled in the art without departing from the scope of the invention as defined by the following claims.

What is claimed is:

1. A method for tattoo removal, comprising:
applying a layer of removal solution to a tattooed region of skin;
regulating a temperature of the layer of removal solution with a temperature control element; and
exposing the layer of removal solution to a light source that produces light at a wavelength between 360-600 nm.

2. The method of claim 1, wherein the removal solution is delivered through a carrier.

3. The method of claim 2, wherein the carrier includes at least one of ethyl alcohol, propylene glycol, lauryl ethers and lauryl esters.

4. The method of claim 1, further comprising:
adjusting a pH level of the removal solution to minimize skin sensitivity.

5. The method of claim 1, wherein the light source includes a plurality of light terminations.

6. The method of claim 1, further comprising:
providing the removal solution in a tissue-covering tray that retains the removal solution against the tattooed region of skin.

7. The method of claim 2, wherein the removal solution contains clindamycin phosphate methyl 7-chloro-6,7,8-trideoxy-6-(1-5 methyl-trans-4-propyl-L-2pyrrolidinecarboxamido)-1-thio-L-threo-α-D-galacto-octopyranoside 2-(dihydrogen phosphate) with a concentration from 0.001%-30% by volume of the carrier.

8. The method of claim 1, further comprising:
combining the removal solution with at least one of a topical antibiotic, topical anesthetic, nicotinic acid, nicotinamide, antimicrobials, salicylic acid, sulfur, retinoids, glycolic acid, tretinoin, and borax.

9. The method of claim 1, further comprising:
regulating the light source through a control panel, the control panel comprising a power source, a timer for controlling a duration of light, and a user interface for displaying a treatment status.

10. The method of claim 1, wherein applying the layer of removal solution further comprises injecting the solution into the tattooed region of the skin at a predetermined depth.

11. The method of claim 1, wherein the removal solution is hydrogen peroxide with a concentration of about 0.3 mM.

12. The method of claim 2, wherein the removal solution is one of hydrogen peroxide, carbamide peroxide and benzoyl peroxide, and ranges from about 0.001%-50% by volume of the carrier.

13. The method of claim 2, wherein the carrier includes at least one of nicotinic acid and nicotinamide in a concentration from about 0.001%-30% by volume of the carrier.

14. The method of claim 2, wherein the removal solution includes one of erythromycin base, tetracycline hydrochloride, and 6-[3-(1-adamantyl)-4-methoxy-phenyl] naphthalene-2-carboxylic acid in a concentration from about 0.001%-30% by volume of the carrier.

15. The method of claim 2, wherein the removal solution comprises tretinoin, 15% carbamide peroxide and 2.5% clindamycin phosphate by volume of the carrier.
